(19) United States
(12) Patent Application Publication
(10) Pub. No.: US 2005/0049282 A1
(43) Pub. Date: Mar. 3, 2005

(54) Process

(75) Inventors: Shalini Andersson, Molndal (SE); Markus Juza, Aarau (CH)

Correspondence Address: WHITE & CASE LLP;PATENT DEPARTMENT, 1155 AVENUE OF THE AMERICAS, NEW YORK, NY, 10036, US

(73) Assignee: AstraZeneca AB, Sodertalje (SE)

(21) Appl. No.: 10/498,944

(22) PCT Filed: Dec. 17, 2002
(86) PCT No.: PCT/SE02/02356

(30) Foreign Application Priority Data
Dec. 18, 2001 (SE) 0104295-1

(51) Int. Cl. A61K 31/4439; C07D 43/02

(52) U.S. Cl. 514/338; 546/272.7

(57) ABSTRACT

The present invention relates to a process for the preparation of certain 2-pyridinyl)methyl]sulfinyl]-1H-benzimidazoles compounds. More specifically it relates to the preparation of an enantiomerically pure or optically enriched enantiomer of either omeprazole, pantoprazole, lansoprazole, or rabeprazole from a mixture containing the same using means for simulated moving bed chromatography.

### FIELD OF THE INVENTION

The present invention relates to a process for the preparation of certain 2-pyridinyl)methyl]sulfinyl]-1H-benzimidazoles compounds. More specifically it relates to the preparation of an enantiomerically pure or optically enriched enantiomer of either omeprazole, pantoprazole, lansoprazole, or rabeprazole from a mixture of the enantiomers using means for simulated moving bed chromatography.

### BACKGROUND OF THE INVENTION AND PRIOR ART

The compound 5-methoxy-2-[[(4-methoxy-3,5-dimethyl-2-pyridinyl)methyl]sulfinyl]-1H-benzimidazole, having the generic name omeprazole, as well as pharmapeutically acceptable salts thereof, are described in EP 5129. Omeprazole is the first member in a family called proton pump inhibitors. Proton pump inhibitors are effective in inhibiting gastric acid secretion, and are consequently useful as antiulcer agents and have revolutionized the treatment of gastrointestinal disorders. Other proton pump inhibitors, such as pantoprazole, lansoprazole, and rabeprazole, are all substituted pyridylsulfinyl benzimidazoles and therefore structurally closely related to omeprazole.

Omeprazole is a sulfoxide and a chiral compound, with the sulfur atom being the stereogenic center. Thus, omeprazole is a racemic mixture of its two single enantiomers, the R- and S-enantiomer of omeprazole.

Pantoprazole, lansoprazole, and rabeprazole, as well as pharmaceutically acceptable salts thereof, are described in U.S. Pat. No. 4,758,579; U.S. Pat. No. 4,628,098; and U.S. Pat. No. 5,045,552, respectively. Traditional chemical synthesis of chiral compounds usually gives the racemic mixture.

In the field of pharmaceutical industry it is an extremely important task to prepare optically pure compounds in order to improve the efficacy of pharmaceuticals per unit dose and to avoid side effects. S-omeprazole for example, has less inter-patient variability of response to treatment than the racemate as well as the corresponding R-isomer.

The separation of a mixture of optical isomers, i.e. optical resolution, has traditionally been performed according to the diastereomer method, the crystallization method, or the enzyme method. In all these methods however, the types of compounds for which optical resolution is feasible are often limited. Resolution of omeprazole using a chiral acyl group, such as mandeloyl, is described in WO 94/27988. Resolution of omeprazole using a crystallization method is described in WO 97/02261. Resolution of omeprazole by bioreduction is described in WO 96/17077 and an enantioselective preparation of omeprazole by biooxidation is described in WO 96/17076.

Chromatography has been recognized as a valuable analytical method, but the potential of preparative chromatography in separating racemates in to their optical antipodes to compete with stereoselective synthesis or traditional resolution has been overseen. In addition, a large quantity of an eluent is needed too and the concentration of the desired compound in an eluate is extremely low, so that much energy and complicated process are required for recovery. Therefore, the development of a method capable of efficient separation in a large quantity has been desired in the art.

Separation of the enantiomers of omeprazole using chromatography is i.a. described in Analyt. Biochem, 136, 293-297 (1984), Allenmark et al.; J. Chromatogr., 456, 323-336 (1988), Marle et al.; J. Chromatogr., 532, 305-319 (1990), Erlandsson et al.; J. Chromatogr., 553, 373-381 (1991), Lindner et al.; J. Chromatogr., 586, 233-248 (1991), Marle et al. Further WO 92/08716 relates to a process for the resolution of certain chiral pyridylmethylsulphinyl-1H-benzimidazoles into their enantiomers.

The concept of simulated moving bed (SMB) was described in the late 1950's, see e.g. U.S. Pat. Nos. 2,957,927; 2,985,589; 3,205,166; 3,291,726 and 3,310,486. Not all stationary phases designed for analytical purposes are equally suited for preparative chromatographic separation of large amounts of a racemate, mainly due to practical and/or economical reasons (availability, cost, mechanical and chemical stability, loadability, etc etc). The chiral stationary phase has to be available in large amounts, with reproducable batch-to-batch properties and at a relatively low cost relative the value of the enantiomers to be separated.

### DESCRIPTION OF THE INVENTION

The present invention relates to a process for chromatograhically resolving enantiomerically pure or optically enriched omeprazole, pantoprazole, lansoprazole, or rabeprazole using means for a simulated moving bed (SMB) system.

Counter-current flows are used efficiently in different chemical processes, such as heat exchanger, extraction, etc. The idea is to implement counter-current adsorption processes involving flows of both the fluid and solid phases in opposite directions. In a true moving bed (TMB) an actual circulation of solid occurs while in an SMB system the solid movement is simulated. A schematic SMB unit is shown in FIG. 1 below and is thus constituted of a number of chromatographic columns, separated by ports where inlet and outlet streams can be fed or collected. The countercurrent solid movement is simulated by periodically shifting the feed and withdrawal points of the unit in the same direction as the mobile phase flow. Four external streams are present, the feed mixture, the desorbent, i.e. the eluent or the mixture of eluents constituting the mobile phase, the extract stream enriched in the enantiomer A, and the raffinated stream enriched in the enantiomer B.

[image]

These streams divide the unit in four sections, section I between the desorbent inlet and the extract port, section II between the latter and the feed inlet, section III between this and the raffinate outlet and section IV between the raffinated port and the desorbent inlet. Each of these sections plays a specific role in the process. The separation is performed in sections II and III, where the less retained enantiomer B must be desorbed and carried by the mobile phase towards the raffinate, while A is retained by the stationary phase and carried towards the extract port through the simulated solid movement. In section I the stationary phase is regenerated by the fresh mobile phase stream and A is conveyed towards the extract port. Finally, in section IV the mobile phase is regenerated by adsorbing the amount of enantiomer B not collected in the raffinate. In this way both the stationary phase and the mobile phase can be recycled to section IV and I, respectively.

The simulated moving bed chromatography for the production of enantimerically pure or optically enriched omeprazole from a mixture comprising the two enantiomers is thus achieved using a set of colums packed with a chromatographic chiral stationary phase (CSP) capable of chiral recognition, ports for the continuous introduction of solvent desorbent (mobile phase) and feed, ports for continuous removal of raffinate (solution containing the less strongly retained enantiomer B), and extract (solution containing the more strongly retained enantiomer A) and a means of recycling fluid through the system. The columns are connected such that the outlet of each column is connected to the inlet of the next column also the outlet of the last column being connected to the inlet of the first column.

The present invention is thus characterized by introducing a solution containing an mixture of the two enantiomers of omeprazole and a desorbing liquid into a plurality of columns containing a CSP therein and having front and rear ends thereof connected to each other endlessly via a fluid passage to circulate a fluid unidirectionally and at the same time drawing out a solution containing one of the separated isomers and another solution containing the other isomer from the columns, wherein a port for introducing a desorbing liquid, a port for drawing out a solution containing a strongly adsorbable optical isomer, i.e. an extract, a port for introducing a solution containing a mixture of optical isomers, and a port for drawing out a solution containing a weakly adsorbable optical isomer, i.e. a raffinate, are arranged on the columns in this order along the direction of fluid and the positions of these ports are successively moved in the direction of fluid flow in the columns intermittently.

The basic operations of an SMB process are adsorption, concentration, desorption, and desorbing liquid recovery and these elements are continuously carried out in the process of the present invention.

Adsorption

The mixture of the two enantiomers of omeprazole is brought into contact with the CSP, so that a strongly adsorbable enantiomer (strongly adsorbable component A) is adsorbed while another weakly adsorbable enantiomer (weakly adsorbable component B) is recovered as a raffinate flow together with the desorbing liquid.

Concentration

The column having the strongly adsorbable component adsorbed thereon is brought into contact with part of the extract described below, so that the weakly adsorbable component remaining on the column is expelled and the strongly adsorbable component is concentrated.

Desorption

The column containing the concentrated strongly adsorbable component is brought into contact with the desorbing liquid, so that the strongly adsorbable component is expelled from the column and recovered together with the desorbing liquid as an extract flow.

Desorbing Liquid Recovery

The column having substantially only the desorbing liquid adsorbed thereon is brought into contact with part of the raffinate flow, so that part of the desorbing liquid contained in the column is recovered as a desorbing liquid recovery.

The flow can be the same or different in the four basic operations indicated above, of which the latter is preferred.

As is indicated in FIG. 1 an SMB system consists of 4 zones. Each zone is defined relative to an injection point and a collection point.
Zone I—between the eluent and extract lines.
Zone II—between the extract and feed lines.
Zone III—between the feed and raffinate lines.
Zone IV—between the raffinate and eluent lines.

The liquid flowing out of zone IV is recycled to zone I. As an example in the case of a binary mixture A+B, A being the less retained component it is possible to choose operating conditions, i.e. flow rates in zones I, II, III, and IV, in order to make A move in one direction, e.g. upwards, and B move in the other direction, e.g. downwards. A and B can thus be recovered respectively in the raffinate and extract streams as pure compounds.

In fact it is extremely difficult to operate a TMB because it involves circulation of a solid adsorbent. This is the reason why another implementation is suitable—the simulated moving bed (SMB). Most of the benefit of counter-current operation can be achieved by using several fixed-bed colums connected in series and an appropriate shift of the injection and collection points. To simulate a counter-current flow, the feed, eluent, extract and raffinate lines are all moved one column (or more) forward in the fluid flow direction at fixed time intervals.

For purposes of this invention, various terms used herein are defined as follows. A “feed mixture” is a mixture containing one or more extract components and one or more raffinate components to be separated by the process, e.g the enantiomers of omeprazole. The term “feed stream” indicates a stream of a feed mixture that passes into the adsorbent, i.e. the CSP, used in the process.

An “extract component” is a compound or class of compounds that is more selectively adsorbed by the adsorbent while a “raffinate component” is a compound or type of compound that is less selectively adsorbed.

The term “desorbent material” shall mean generally a material capable of desorbing an extract component from the adsorbent.

The term “raffinate stream” or “raffinate output stream” means a stream in which a raffinate component is removed from the apparatus.

The term “extract stream” or “extract output stream” shall mean a stream in which an extract material that has been desorbed by a desorbent material is removed from the apparatus.

The term “compound(s) of the present invention” shall mean enantiomerically pure omeprazole, pantoprazole, lansoprazole, or rabeprazole.

Typically at least a portion of the extract stream and the raffinate stream are passed to separation means, normally evaporators or crystallizers but possibly a fractional distillation column, wherein at least a portion of desorbent material is recovered. This will also produce an extract product and possibly a raffinate product.

Continuous SMB systems have numerous advantages over batch-type processes. An SMB process produces a constant uniform composition product. It is flexible and the recovery and purity of the product can normally be adjusted. An SMB process apparatus comprises many serially-connected columns with intermediate points for the appropriate addition or removal of feed, extract, desorbent and raffinate streams. Cyclic advancement of the input and output streams through the apparatus can be accomplished by a multiple valve manifold system. In these simulated moving bed systems the adsorbent is usually divided between eight or more columns. The configuration of the eight columns may not necessary be 2+2+2+2, as is schematically shown in FIG. 1. A column configuration of 5+1+3+3, or any other distribution of colums including those with variable-lengths chromatographic zones, is also feasible. Equipment utilizing these SMB principles can vary in size. The most difficult part is finding an effective adsorbent/desorbent system and suitable conditions.

In simulated moving bed adsorptive separation processes, which are generally operated continuously at substantially constant pressures and temperatures that insure liquid phase, the desorbent material must be judiciously selected to satisfy many criteria. First, the desorbent material should displace an extract component from the adsorbent with reasonable mass flow rates.

Secondly, desorbent materials must be compatible with the particular adsorbent and the particular feed mixture. More specifically, they must not reduce or destroy the capacity of the adsorbent or selectivity of the adsorbent for an extract component with respect to a raffinate component. Additionally, desorbent materials should not chemically react with or cause a chemical reaction of either an extract component or a raffinate component.

Thirdly, desorbent materals should consist of a single solvent, or a binary mixture of solvents and complex solvent mixtures should be avoided, if possible.

Finally, desorbent materials should be readily available and reasonable in cost. The desorbent material of the mobile phase will have to be selected in each instance based upon the above criteria and its performance with the stationary phase.

As discussed above, colums with variable-lengths chromatographic zones are also feasible to be used in the present invention. Variable-lengths chromatographic zones are achievable if the shifting of different injection and draw-off points or a column, or column section, is carried out at different times instead of simultaneously. If that is the case, it should be noted that at the end of a cycle the system has regained its initial position.

It is advantageous to use a liquid as an eluent, but it is also possible to operate with a subcritical fluid.

The range of pressures in which the separations of products are carried out can be between 0.1 and 50 MPa and preferably between 0.5 and 30 MPa. The temperature in the columns is generally between 0° C. and 100° C.

Chiral Stationary Phase (CSP)

Prerequisites for scaling up a chromatographic analytical chiral separation into an SMB system is that the CSP is available in large amounts, with reproducible batch-to-batch properties and at a relatively low cost with respect to the value of the enantiomers to be separated. If this is fulfilled then the economical feasability of the SMB process will be dictated by the key properties of the CSP namely selectivity, loading capacity and efficiency. These parameters later have an impact on the size of the unit and the achievable specific productivity of the process per unit mass of stationery phase. Other important issues are chemical stability, compatible mobile phases, and solubility of the enantiomers. All these characteristics have to be properly taken into account when selecting the CSP for an SMB system.

The CSPs most commonly used in enantioselective chromatography and SMB applications can be grouped as follows (see table 1 for some examples of chemical structure, commercial name and supplier).
i. cellulose derivatives (e.g. esters or carbamates, preferably deposited on silica);
ii. tartrate phases;
iii. π-acidic and π-basic CSPs (Pirkle phases);
iv. amylose derivatives (e.g. esters or carbamates, prferably deposited on silica)
v. polyacrylamide phases.

vi. others

TABLE 1
Structure and properties of commercial chiral stationary phases

| Structure of CSP | chiral selector | trade name |
|---|---|---|
| 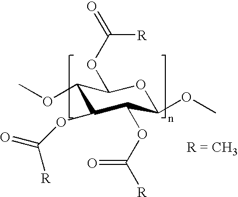 | micro- crystalline cellulose- triacetate | MCTA or CTA-I |
| 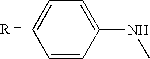 | cellulose tris(phenyl- carbamate) | Chiracel OJ |
| 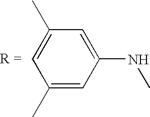 | cellulose tris(3,5-di- methylphenyl- carbamate) | Chiracel OD |
| 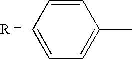 | cellulose tribenzoate | Chiracel OB |
| 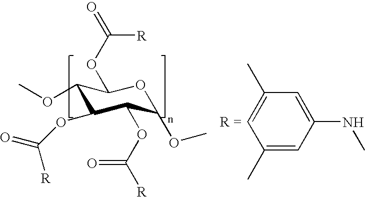 | amylose tris(3,5-di- methyl- phenylcarbamate) | Chiralpak AD |
| 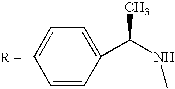 | amylose tris[(S)-methyl- benzylcarbamate] | Chiralpak AS-V |
| 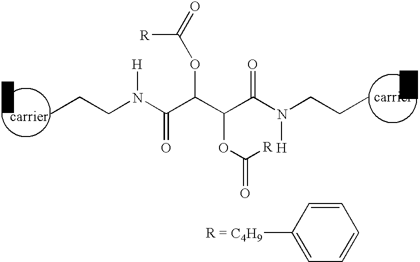 | O,O′-bis(4-tert-butyl- benzoyl)-N,N′-di- allyl-L-tartar- diamide | Kromasil CHI-TBB |
| 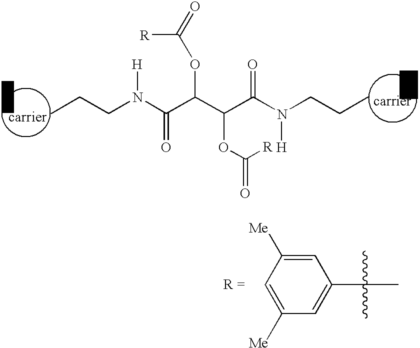 | O,O′-bis(dimethyl-ben- zoyl)-N,N′-di- allyl-L-tartar- diamide | Kromasil-CHI-DMB |
| 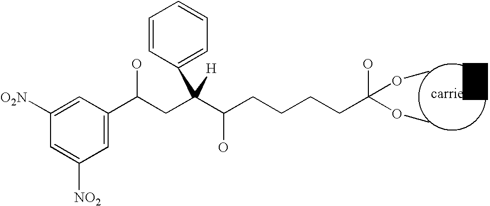 | 3,5-dinitrobenzoyl-phenyl- glycine (either ionic or covalent bonding) | DNBPG |

Special care should be given to the following regarding stationary/mobile phase system used in SMB: a) retention time; b) enantioselectivity; c) loading capacity; d) productivity; e) eluent consumption; f) avoiding complex eluent mixtures or buffer additives.

Cellulose Based CSPs

Table 2 shows an extensive data set of polysaccharide based stationary phases screened for chiral resolution of omeprazole using 10 μm and 20 μm stationary phases. 20 μm stationary phases on silica particles is considered as the material of choice for scaling-up enantioselective preparative scale chromatographic separations since they combine low back pressure and sufficient resolution at high flow rates. It can be observed that most cellulosic stationary phases show relatively high k′ values for the two enantiomers of omeprazole and some even show no chiral recognition ability. Long retention times lead in general to high cycle times and high eluent consumption. Surprisingly the cellulose based CSPs were found not to be the material of choice for scaling-up due to problem with scale-up and long retention times.

Tartrate CSPs

Table 5 shows an extensive data set of Tartrate CSPs screened for chiral resolution of omeprazole. Kromasil-CHI TBB, 16 μm appears to be the most promising CSP and additional data for this CSP is shown in Table 6. Surprisingly the tartrate CSPs were found not to be the material of choice for scaling-up due to complex and expensive solvent mixtures as the desorbent material. The peak shapes of these systems were further not satisfying.

π-Acidic and π-Basic CSPs

Table 7 shows data set of π-acidic and π-basic CSPs screened for chiral resolution of omeprazole. Surprisingly the π-acidic and π-basic CSPs were found not to be the material of choice for scaling-up due to complex and expensive solvent mixtures as the desorbent material and not sufficient loading capacity.

Amylose Based CSPs

The tris(3,5-dimethylphenyl carbamate) derivative of amylose has been commercialized under the name Chiralpak AD, the tris[(S)-methylbenzylcarbamate] has been named Chiralpak AS. The latter derivative, as well as providing polar, polarizable sites, also contributes another chiral center. The (S)-configured methyl group is also available as well the (R,S) and (R)-derivative. Table 3 shows an extensive data set using 20 μm stationary Chiralpak AD. It should be noted that the results obtained for 10 μm particle size stationary phase could not be reproduced with Chiralpak AD 20 μm particle size material. Table 4 shows an extensive data set using 20 μm stationary Chiralpak AS.

It was surprisingly found that the order in which the two enantiomers of omeprazole eluted reversed when going from 10 μm particle size to 20 μm particle size Chiralpak AS and EtOH/IPA 30/70 as the desorbent material. Using these conditions S-(−)-omeprazole is the more retained component and will elute in the extract, while R-(+)-omeprazole is the less retained component and will elute in the raffinate.

The enantiomeric excess (e.e.) in the raffinate and/or extract is usually above 90%, preferably above 95% or even more preferably above 98%. However since it is possible to improve the e.e. by a subsequent crystallization step, an e.e. of 60% in the raffinate and/or extract is sufficient to be able to prepare the compounds of the present invention. It is also possible to improve the e.e. by converting a compound of the present invention into a base addition salt thereof and crystallize the salt.

In one embodiment of the present invention the enantiomeric excess (e.e.) in the raffinate and/or extract is 60% and above, preferably above 70% or even more preferably above 80%. The e.e. is thereafter improved by a subsequent crystallisation step, optionally with a pre-conversion of the compound into a base addition salt.

The racemic mixture, i.e. a mixture containing equal amounts of the two enantiomers, is the most easily accessible mixture using traditional chemical synthesis. However use of enantioselective chemical synthesis and enzymatic synthesis may give other ratios of the two enantiomers. Both the racemic mixture and a mixture with any other ratio of the two enantiomers than a 50:50 ratio are suitable for the present invention. It is preferred to use the racemic mixture for practical reasons.

The process of the present invention is preferably used to isolate one of the enantiomers of either omeprazole, pantoprazole, lansoprazole, or rabeprazole. The other enantiomer might be discarded but is preferably taken through a racemisation procedure that generates a mixture containing both enantiomers with thereafter can be purified according to the present invention. Such a procedure is also within the scope of the present invention.

Stability of Omeprazole Under Chromatographic Conditions

It has previously been reported that alcoholic solution of omeprazole is not stable in room temperature and in daylight. There is thus a risk that one or several decomposition products might co-elute with one of the enantiomers. However, the addition of 0.1% diethylamine, or any other similar organic amine, stabilizes a solution of omeprazole in methanol to a sufficient degree. We have now surprisingly found that omeprazole can be resolved into its two enantiomers using means for SMB and ethanol as the mobile phase.

Simulation of SMB Operating Parameters

To design and optimize a SMB separation, NOVASEP (Nancy, France) has developed a procedure based on the theory of multicomponent chromatography. This procedure includes mainly two steps:
1. Measuring the characteristics of the stationary phase. These data can include: competitive adsorption isotherms, Van-Deemter curve which gives HETP (the height equivalent to a theoretical plate) vs. the mobile phase velocity, the relationship between pressure drop and the mobile phase velocity.
2. The data measured in the previous steps are processed by NOVASEP's simulation software “softSMB”, or any other suitable software such as LicoHELP, which will estimate the operating conditions and SMB parameters.

SoftSMB can be used to predict operating conditions and SMB parameters of the present invention. Predicted productivity and eluent consumption is shown in FIGS. 5 and 6. The composition of the eluent of the present invention can be either isocratic, or a composition gradient. It is also recommended to add small amounts of an organic amine to stabilize the compounds of the present invention.

### EXAMPLES

The following common abbreviations are used.

| | |
|---|---|
| AA | acetic acid |
| ACN | acetonitrile |
| DEA | diethylamine |
| EtOAc | ethyl acetate |
| EtOH | etanol |
| IH | isohexane |
| IPA | isopropyl alcohol |
| MeCl | dichloromethane |
| MeOH | methanol |
| MTBE | methyl tert. butyl ether |
| n-Hex | n-hexane |
| TEA | triethylamine |

### Example 1

Stationary and mobile phases were screened for separation of Omeprazole into its enantiomers. Result is given below in Table 2.

TABLE 2
Systematic screening of stationary (cellulose based) and mobile phases
for the separation of omeprazole into its enantiomers.

| | | | | | | | |
|---|---|---|---|---|---|---|---|
| | Solute | | | | | | |
| Mobile phase | in | k′₁ | k′₂ | α | Rₛ | Plates₁ | Plates₂ |

Column: Chiralpak AD

| IH/IPA 90/10 | IPA | 7.40 | 8.98 | 1.21 | — | 268 | |
|---|---|---|---|---|---|---|---|
| IH/IPA 70/30 | IPA | 1.37 | 1.71 | 1.25 | — | 361 | |
| Ethanol | IPA | 1.34 | 1.93 | 1.44 | 1.57 | 803 | 772 |
| EtOH/IH 80/20 | IPA | 1.11 | 1.63 | 1.46 | 1.68 | 951 | 972 |
| EtOH/IH 80/20 + 0.2TEA | IPA | 1.60 | 1.99 | 1.24 | — | — | — |
| Methanol | IPA | 1.24 | 1.24 | 1.00 | — | — | — |

| | | | | | | |
|---|---|---|---|---|---|---|
| Acetonitrile | IPA | 4.97 | 4.97 | 1.00 | — | 230 |

Column: Chiralpak AS

| IH/IPA 90/10 | IPA | 15.13 | | | — | — | — |
|---|---|---|---|---|---|---|---|
| IH/IPA 70/30 | IPA | 2.90 | 5.80 | 2.00 | 2.55 | 670 | 262 |
| Ethanol | IPA | 0.54 | 0.78 | 1.43 | 1 | 933 | 696 |
| IH/EtOH 70/30 | IPA | 1.92 | 2.97 | 1.55 | 3.02 | 1947 | 1332 |
| IH/EtOH 65/35 | IPA | 1.61 | 2.51 | 1.56 | 2.72 | 1760 | 1181 |
| IH/EtOH 65/35 + 0.2TEA | IPA | 1.11 | 1.51 | 1.68 | 2.46 | 2124 | 682 |
| Methanol | IPA | 0.49 | 0.49 | 1.00 | — | 560 | |
| Acetonitrile | IPA | 0.93 | 1.22 | 1.32 | 1.3 | 1264 | 1387 |

Column: Chiralcel OA

| | | | | | | | |
|---|---|---|---|---|---|---|---|
| IH/IPA 90/10 | IPA | 6.92 | 7.99 | 1.15 | — | — | — |
| IH/IPA 80/20 | IPA | 2.59 | 2.95 | 1.14 | — | — | — |

| | | | | | | |
|---|---|---|---|---|---|---|
| Ethanol | IPA | 0.20 | 0.20 | 1.00 | — | 1790 |

Column: Chiralcel OB

| | | | | | | |
|---|---|---|---|---|---|---|
| IH/IPA 90/10 | IPA | 5.17 | 5.17 | 1.00 | — | 22 |
| IH/IPA 80/20 | IPA | 1.83 | 1.83 | 1.00 | — | 27 |
| Ethanol | IPA | 0.17 | 0.17 | 1.00 | — | 608 |

Column: Chiralcel OD

| IH/IPA 90/10 | IPA | 7.57 | 9.93 | 1.31 | 1.81 | 1012 | 1110 |
|---|---|---|---|---|---|---|---|
| IH/IPA 80/20 | IPA | 2.83 | 3.69 | 1.30 | 1.7 | 1072 | 1142 |
| IH/IPA 70/30 | IPA | 1.54 | 2.34 | 1.33 | 1.49 | 1054 | 1107 |
| Ethanol | IPA | 0.45 | 0.45 | 1.00 | — | 570 | |
| Methanol | IPA | 0.52 | 0.52 | 1.00 | — | 1023 | |
| Acetonitrile | IPA | 1.56 | 1.56 | 1.00 | — | 523 | |
| IH/EtOH 70/30 | IPA | 1.00 | 1.27 | 1.27 | 1.24 | 1470 | 1446 |

Column: Chiralcel OG

| | | | | | | | |
|---|---|---|---|---|---|---|---|
| IH/IPA 90/10 | IPA | tᵣ > 60′ | | | — | — | — |
| IH/IPA 80/20 | IPA | 7.61 | 9.60 | 1.26 | 1.58 | 831 | 1024 |
| Methanol | IPA | 0.49 | 0.59 | 1.20 | 0.82 | 2870 | 2336 |

Column: Chiralcel OJ

| | | | | | | | |
|---|---|---|---|---|---|---|---|
| IH/IPA 90/10 | IPA | 4.80 | 7.05 | 1.47 | 2.55 | 888 | 1050 |
| IH/IPA 80/20 | IPA | 1.73 | 2.33 | 1.35 | 1.49 | 928 | 888 |
| IH/IPA 70/30 | IPA | 0.93 | 1.19 | 1.28 | 0.93 | 939 | 776 |
| Ethanol | IPA | 0.16 | 0.16 | 1.00 | — | — | — |
| Methanol | IPA | 0.15 | 0.15 | 1.00 | — | — | — |
| EtOH/IH 10/90 | IPA | 4.00 | 4.83 | 1.21 | 0.75 | 766 | 251 |

### Example 2

Chiralpak AS; 20 μm particle size, is optimized for SMB. Results are given in Table 4.

TABLE 4
Chiralpak AS; 20 μm particle size

| mobile phase | dissolved in | Rₜ₁ [min] | Rₜ₂ [min] | α |
|---|---|---|---|---|
| IH/EtOH 30/70 | IPA | 8.296 | 13.670 | 1.81 |
| IH/EtOH 35/65 | IPA | 7.423 | 11.916 | 1.78 |
| IH/EtOH/DEA | IPA | 7.362 | 11.685 | 1.75 |
| 30/70/0.2 | | | | |
| ACN | IPA | 5.731 | 7.532 | 1.44 |
| EtOH | IPA | 4.369 | 5.856 | 1.54 |
| EtOH/IPA 30/70 | IPA | 5.487 | 8.902 | 1.92 |
| EtOH/IPA 35/65 | IPA | 5.330 | 8.463 | 1.85 |

column: Chiralpak AS; 120 Å, dₚ= 20 μm, 250 mm × 4.6 mm, T = 25° C., detection @ 334 nm, injected volume: 20 μL.

The similarities and differences of the solvent systems described in Table 4 have been analyzed in regard to productivity and solvent consumption in detail (cf. Example 8).

### Example 3

Additional stationary and mobile phases are screened for the separation of of omeprazole into its enantiomers. Results are given in Table 5.

TABLE 5
Systematic screening of stationary (tartrate based) and mobile
phases for the enantiomer separation of Omeprazole

| | | | | | | | |
|---|---|---|---|---|---|---|---|
| | Solute | | | | | | |
| Mobile phase | in | k′₁ | k′₂ | α | Rₛ | Plates₁ | Plates₂ |

Column: Kromasil-CHI-DMB

| | | | | | | | |
|---|---|---|---|---|---|---|---|
| IH/IPA 90/10 | IPA | 4.23 | 4.78 | 1.13 | 1.05 | 1952 | 1603 |
| IH/EtOAc 50/50 | EtOAc | 4.28 | 5.61 | 1.31 | 2.46 | 1950 | 1918 |
| IH/Dioxan 70/30 | EtOAc | 3.16 | 3.75 | 1.19 | 1.7 | 2678 | 2479 |
| IH/MeCl 50/50 | EtOAc | tᵣ > 60′ | | | — | — | — |
| IH/MTBE 70/30 | EtOAc | tᵣ > 60′ | | | — | — | — |
| IH/EtOAc/IPA 80/15/5 | EtOAc | 4.47 | 5.41 | 1.21 | 1.97 | 2628 | 2373 |
| IH/EtOAc/IPA 60/35/5 | EtOAc | 2.57 | 3.13 | 1.22 | 1.92 | 2985 | 2727 |
| IH/MTBE/IPA 50/35/15 | EtOAc | 1.79 | 2.11 | 1.18 | 1.21 | 2133 | 1752 |
| IH/MTBE/IPA 50/40/10 | EtOAc | 3.06 | 3.77 | 1.23 | 1.74 | 1996 | 1777 |
| IH/MeCl/IPA 60/30/10 | EtOAc | 0.59 | 0.59 | 1.00 | — | — | — |

Column: Kromasil-CHI-TBB

| | | | | | | | |
|---|---|---|---|---|---|---|---|
| IH/IPA 90/10 | IPA | 3.93 | 5.32 | 1.35 | 2.71 | 1976 | 1851 |
| EtOH | IPA | — | — | — | — | — | — |
| IH/Dioxan 70/30 | EtOAc | 3.24 | 4.67 | 1.44 | 2.91 | 1454 | 1913 |
| IH/MeCl 50/50 | EtOAc | 7.65 | 9.06 | 1.18 | — | — | — |
| IH/MeCl/IPA 60/30/10 | EtOAc | 0.25 | 0.25 | 1.00 | — | — | — |
| IH/MeCl/IPA 80/15/5 | EtOAc | 2.49 | 3.24 | 1.30 | 1.70 | 1098 | 1380 |
| IH/MeCl/IPA 70/25/5 | EtOAc | 1.20 | 1.52 | 1.26 | 1.37 | 1531 | 1918 |
| IH/MTBE 70/30 | EtOAc | — | — | — | — | — | — |
| IH/MTBE/IPA 50/35/15 | EtOAc | 1.72 | 2.48 | 1.44 | 2.51 | 1667 | 1604 |
| IH/MTBE/IPA 50/40/10 | EtOAc | 3.02 | 4.54 | 1.50 | 3.04 | 1488 | 1430 |
| IH/MTBE/IPA 50/40/10 + 0.1% AA | EtOAc | 3.39 | 4.99 | 1.47 | 3.51 | 2138 | 1979 |
| IH/MTBE/IPA 50/40/10 + 0.2% | EtOAc | 3.40 | 5.05 | 1.48 | 3.55 | 2111 | 1936 |
| TEA | | | | | | | |
| IH/MTBE/IPA 50/40/10 + 0.15% AA | EtOAc | 3.19 | 4.67 | 1.46 | 3.48 | 2248 | 2069 |
| IH/MTBE/IPA 45/45/10 + 0.1 TEA | EtOAc | 3.68 | 5.52 | 1.50 | 2.32 | 718 | 842 |
| IH/EtOAc 70/30 | EtOAc | — | — | — | — | — | — |
| IH/EtOAc 60/40 | EtOAc | 8.36 | 13.10 | 1.57 | 2.95 | 572 | 1143 |
| IH/EtOAc 50/50 | EtOAc | 3.50 | 5.35 | 1.53 | 2.62 | 730 | 1157 |
| IH/EtOAC/IPA 80/15/5 | EtOAc | 4.44 | 6.41 | 1.44 | 2.91 | 1381 | 1474 |
| IH/EtOAc/IPA 75/20/5 | EtOAc | 3.72 | 5.11 | 1.37 | — | — | — |
| IH/EtOAc/IPA 75/20/5 | IPA | 3.17 | 4.30 | 1.36 | 2.97 | 2302 | 2606 |
| IH/EtOAc/IPA 75/20/5 | IPA + TEA | 3.37 | 4.54 | 1.35 | 2.35 | 1244 | 1921 |
| IH/EtOAc/IPA | IPA + TEA | 3.36 | 4.63 | 1.38 | 3.70 | 3369 | 3364 |
| 75/20/5 + 0.05AA, 0.1 TEA | | | | | | | |
| IH/EtOAc/IPA | EtOAc | 4.20 | 5.87 | 1.40 | 4.39 | 4049 | 3931 |
| 75/20/5 + 0.05AA, 0.1 TEA | | | | | | | |
| IH/EtOAc/IPA 60/35/5 | EtOAc | 2.67 | 3.84 | 1.44 | 2.80 | 1553 | 1714 |
| IH/EtOAc/IPA 60/35/5 + 0.1 TEA | EtOAc | 2.66 | 3.85 | 1.45 | 3.02 | 1821 | 1867 |

### Example 4

Kromasil-CHI TBB, 16 μm particle size, is evaluated as a potential CSP. The results are given in table 6.

TABLE 6
Kromasil-CHI TBB, 16 μm particle size

| mobile phase | dissolved in | Rₜ₁ [min] | Rₜ₂ [min] | α |
|---|---|---|---|---|
| Ethanol | MeOH | 3.428 | — | 1.00 |
| IH/IPA 90/10 | MeOH | 12.584 | 15.857 | 1.29 |
| IH/EtOH 90/10 | MeOH | 9.385 | 10.707 | 1.17 |

### Example 5

(S)-α-Burke 2, 10 μm particle size, is screened as a potential CSP. The result is given in Table 7.

TABLE 7
(S)-α-Burke 2, 10 μm particle size

| mobile phase | dissolved in | Rₜ₁ [min] | Rₜ₂ [min] | α* |
|---|---|---|---|---|
| CH₂Cl₂/MeOH 95/5 | MeOH | 5.061 | 5.709 | 1.19 |
| Methanol | MeOH | 3.873 | 4.172 | 1.13 |

### Example 6

Chiralpak AD; 20 μm particle size, is optimized for SMB. Results are given in Table 3.

TABLE 3
Chiralpak AD; 20 μm particle size

| mobile phase | dissolved in | Rₜ₁ [min] | Rₜ₂ [min] | α |
|---|---|---|---|---|
| n-Hex/EtOH/ACN/DEA | MeOH | 3.733 | 4.274 | 1.26 |
| 53/31/16/0.1 | | | | |
| n-Hex/EtOH/ACN/DEA | MeOH | 4.119 | 4.999 | 1.35 |
| 69.7/20/10.3/0.1 | | | | |
| n-Hex/EtOH/ACN/DEA | MeOH | 4.864 | 6.641 | 1.55 |
| 79.8/3113.3/6.9/0.1 | | | | |
| n-Hex/EtOH/ACN/DEA | MeOH | 7.386 | 12.749 | 1.93 |
| 88.6/7.5/3.9/0.1 | | | | |
| Ethanol | MeOH | 4.632 | — | 1.00 |
| EtOH/IH 20/80 | MeOH | 4.402 | — | 1.00 |

### Example 7

Simulation of SMB parameters using NOVASEP's simulation software softSMB.

The competitive adsorption isotherms have been determined using the procedures developed by NOVASEP; it was found that the experimental data fit well into the modified Langmuir competitive isotherm model. The model can be written as:

$${n}_{i} = λ \cdot {c}_{i} + \frac{{\overline{N}}_{i} \cdot {K}_{i} \cdot {c}_{i}}{1 + {\sum }_{j = 1}^{2} {K}_{j} \cdot {c}_{j}}$$

In this equation ni and ci are the adsorbed and the fluid phase concentration, respectively; λ is a dimensionless constant; Kᵢ is the equilibrium constant of the i-th component, which accounts for the overload effects; the upper limit of Nᵢ is given by the saturation capacity and measures the amount of sample which can be loaded onto the column. The isotherm data for Chiralpak AS and various eluent compositions are summarized in the following table.

TABLE 8
Derived isotherms for various mobile phase combinations

| Experiment | mobile phase | λ | N₁K₁ | N₂K₂ | \overline{N}ᵢ |
|---|---|---|---|---|---|
| A | IH/EtOH 30/70 | 1.0 | 0.8612 | 3.016 | 28 |
| B | IH/EtOH 35/65 | 1.5 | 0.8110 | 2.613 | 24 |
| C | IH/EtOH/DEA | 1.6 | 0.686 | 2.420 | 28 |
| | 30/70/0.2 | | | | |
| D | ACN | 1.1 | 0.532 | 1.254 | 34 |
| E | EtOH | 0.5 | 0.585 | 1.182 | 44 |
| F | EtOH/IPA 30/70 | 1.3 | 0.234 | 1.664 | 38 |
| G | EtOH/IPA 35/65 | 1.2 | 0.271 | 1.528 | 30 |

column: Chiralpak AS; 120 Å, dₚ = 20 μm, 250 mm × 4.6 mm, T = 25° C., detection @ 334 nm.

### Example 8

A comparison involving the daily productivity for a given SMB system with eight columns (10.1 cm×4.8 cm ID; configuration 2-2-2-2) and the eluent consumption per day shows the following characteristics (cf. FIG. 5):

[image]

### Example 10

The modeling/simulation results were confirmed by comparing experimental peak retention times and calculated retention times obtained from overloaded injections. In all cases, the agreements are reasonably good, for a comparison see the following table:

TABLE 9
Validation of simulation results

| experiment | concentration | injected | Rt₁ [min] | Rt₁ [min] | Rt₂ [min] | Rt₂ [min] |
|---|---|---|---|---|---|---|
| No | omeprazole [g/L] | Vol. [μL] | measured | calculated | measured | calculated |
| 1 | analytical | 10 | 5.49 | 5.31 | 8.90 | 8.90 |
| 2 | 24.48 | 10 | 5.43 | 5.25 | 8.81 | 8.63 |
| 3 | 24.48 | 20 | 5.41 | 5.44 | 8.72 | 8.53 |
| 4 | 24.48 | 50 | 5.36 | 5.44 | 8.47 | 8.33 |
| 5 | 24.48 | 100 | 5.30 | 5.44 | 8.20 | 8.14 |
| 6 | 24.48 | 200 | 5.19 | 5.44 | 7.86 | 7.92 |
| 7 | 24.48 | 250 | 5.19 | 5.44 | 7.75 | 7.84 |

column: Chiralpak AS, 120 Å, dₚ = 20 μm, 250 mm × 4.6 mm, T = 25° C., detection @ 334 nm, injected volumes: see table.

### Example 11

A suitable SMB system is used with Chiralpak AS as the CSP and with the parameters indicated below in Table 11. Optical purity of the extract and raffinate are also indicated in Table 11.

TABLE 11
Operating parameters, productivity and purities

| Operating parameters | Feed concentration |
|---|---|
| (solvent system EtOH/IPA 30/70) | 27.0 g/L |
| Recycling flow (Zone 1) [mL/min] | 273.0 |
| Extract flow [mL/min] | 108.10 |
| Feed flow [mL/min | 45.00 |
| Eluent flow [mL/min] | 118.27 |
| Raffinate flow [mL/min] | 55.17 |
| Purity extract [%] | 99.79 |
| Purity raffinate [%] | 99.94 |
| Productivity [g/day] | 1740 |

### Example 12 and 13

In Examples 12 and 13 the following general procedure was used. The columns (diameter of 25.4 mm) of the LICOSEP Lab Bex was packed using C Pak AS 20 μm (Daicel). To reach a bed length of about 11 cm, 30 g of chiral stationary phase per column were used. Either a 2-2-2-2 or s 3-3-3-3 configuration was used, the mobile phase was pure ethanol and the feed concentration was fixed to 10 g omeprazole/liter.

| | Number | | | | | | Switching | Zone flow rates |
|---|---|---|---|---|---|---|---|---|
| | of | Q feed | Q elu | Q ext | Q raf | Q rec | Period | and |
| Example # | columns | (mL/min) | (mL/min) | (mL/min) | (mL/min) | (mL/min) | (min) | purities |
| 12 | 12 | 5.3 | 11.8 | 10.6 | 6.5 | 39.8 | 2.10 | Zone I: 2.39 L/h |
| | | | | | | | | Zone II: 1.75 L/h |
| | | | | | | | | Zone III: 2.07 L/h |
| | | | | | | | | Zone IV: 1.68 L/h |
| | | | | | | | | Extract purity: |
| | | | | | | | | 98.2% e.e. |
| | | | | | | | | Raffinate purity: |
| | | | | | | | | 99.6% e.e. |
| 13 | 8 | 7.5 | 18.7 | 15.4 | 10.8 | 62.0 | 1.40 | Zone I: 3.72 L/h |
| | | | | | | | | Zone II: 2.82 L/h |
| | | | | | | | | Zone III: 3.25 L/h |
| | | | | | | | | Zone IV: 2.60 L/h |
| | | | | | | | | Extract purity: |
| | | | | | | | | 98.0% e.e. |
| | | | | | | | | Raffinate purity: |
| | | | | | | | | 99.1% e.e |

Using the conditions of Examples 12 and 13, above 98% e.e. and with the same bed length, the productivity would be 7.71 g/h for an SMB 8×50 and 133.9 g/h (1071 kg/year) for an SMB 8×200 for each enantiomer. This corresponds to a specific productivity of 438 g mixture of the enantiomers/kg CSP/24 h. Thus, according to one embodiment of the present invention the specific productivity of for the production of enantiomerically pure or optically enriched enantiomer of omeprazole is above 400 g mixture of the enantiomers/kg CSP/24 h, preferably 300 to 500 g, more preferably about 400 to 500 g and even more preferably about 440 g mixture of the enantiomers/kg CSP/24 h.

1. A process for the preparation of an enantiomerically pure or optically enriched enantiomer of a benzimidazole compound selected from the group consisting of omeprazole, pantoprazole, lansoprazole and rabeprazole from a mixture of the enantiomers, wherein the process comprises resolving the enantiomerically pure or optically enriched enantiomer from the mixture by simulated moving bed chromatography.

2. The process according to claim 1, wherein the mixture consists of the enantiomers of omeprazole.

3. The process according to claim 1, wherein the process further comprises a stationary phase comprising amylose tris(S)-methyl-benzycarbamate.

4. The process according to claim 3, wherein the particle size of the chiral stationary phase is 20 μm.

5. The process according to claim 1, wherein the process further comprises a mobile phase comprising ethanol.

6. The process according to claim 1, wherein the process produces an extract consisting essentially of S-(−)-omeprazole.

7. The process according to claim 1, wherein the process produces separation products selected from the group consisting of an extract and a raffinate, and wherein the separation product is characterized by an enantiomeric excess of 98% or above.

8. A process for resolving an enantiomerically pure or optically enriched enantiomer of omeprazole from a mixture of the enantiomers by simulated moving bed chromatography, wherein the process comprises a chiral stationary phase comprising amylose tris(S)-methyl-benzycarbamate in 20 μm particle size and a mobile phase comprising ethanol, and wherein the process produces an extract consisting essentially of S-(−)-omeprazole.

9. The process according to claim 8, wherein the extract is characterized by an enantiomeric excess of 95% or above.

10. The process according to claim 8, wherein the extract is characterized by an enantiomeric excess of 98% or above.

11. The process according to claim 8, wherein more than 400 g of the enantiomeric mixture/kg CSP/24 h is resolved.
